(19) United States
(12) Patent Application Publication
(10) Pub. No.: US 2006/0120980 A1
(43) Pub. Date: Jun. 8, 2006

(54) Novel dermatological composition using bio-activating organocatalysts

(76) Inventor: James J. Eberl, Hilton Head Island, SC (US)

Correspondence Address: COLEMAN SUDOL SAPONE, P.C., 714 COLORADO AVENUE, BRIDGE PORT, CT, 06605-1601, US

(21) Appl. No.: 11/292,227

(22) Filed: Dec. 1, 2005

Related U.S. Application Data
(60) Provisional application No. 60/632,479, filed on Dec. 2, 2004.

(51) Int. Cl. A61K 8/49

(52) U.S. Cl. 424/62

(57) ABSTRACT

The invention provides novel dermatological compositions and related methods useful in the activation of skin growth factors and growth receptors. Compositions of the invention act upon follicle cells and other skin targets to induce hair growth, facilitate dermal cell repair, and enhance skin health. Compositions comprise a bio-activating organocatalyst in a pharmaceutically acceptable carrier, adapted for use on an animal's skin or hair.

### RELATED APPLICATIONS

This application claims the benefit of provisional application U.S. 60/632,479, filed Dec. 2, 2004, the entirety of which is incorporated by reference herein.

### FIELD OF THE INVENTION

The invention relates to novel dermatological compositions and related methods which utilize bio-activating organocatalysts useful in the activation of skin growth factors and growth receptors. Compositions of the invention act upon follicle cells and other skin targets to induce hair growth, facilitate dermal cell repair, and enhance skin health.

### BACKGROUND OF THE INVENTION

Free radical formation has long been associated with detrimental physical occurrences including tissue damage. While transition metal-containing compositions and enzymes have been used in the treatment of skin disorders and in hair growth stimulation, such compositions have not utilized controlled redox reactions to achieve desired dermatological clinical endpoints. Our previous invention made use of such controlled redox reactions to achieve desired dermatological clinical endpoints, including hair growth and skin restoration.

U.S. Pat. No. 5,888,522 discloses peptone digests complexed with one or more ionic transition metals, such as copper, indium, tin, zinc, or the salts thereof, that are alleged to be useful in treating a variety of skin disorders. Japanese Patent No. 2002332217 to Fujii, et al. discloses hair stimulating compositions containing coenzyme Q.

U.S. Pat. No. 6,544,531 discloses that: (1) retinol or vitamin A alcohol is useful in the reduction of fine lines, wrinkles, and mottled hyperpigmentation in skin; (2) hydroxy acids, and particularly alpha-hydroxy acids, are useful in increasing the clarity of the skin surface, increasing cellular turnover, and increasing skin radiance and smoothness; and (3) ascorbic acid has skin permeation and collagen synthesis activity. U.S. Pat. No. 6,544,531 discloses compositions which include: a retinoid and preferably retinol; a dermatologically active acid; and a volatile base, such as, for example, ammonium hydroxide.

All living organisms and their processes can be described by two chemical reaction rate constants as follows:
(death) k₁<==========================>k₂ (growth)

Human biology is a dynamic cellular process all controlled by chemical reaction rates! At equilibrium, the reaction rate constants are equal while in youth k₂ is larger than k₁, while in old age, k₁ exceeds k₂.

Some cellular processes are very rapid, e.g., mucous membranes turn over every 24 to 48 hours. Others can be slow, e.g., prostate cells require as long as one year for replacement. Even bone replaces itself where the turnover rate is at least a couple of years. Thus, every living tissue is in a dynamic state of chemical change via chemical reactions.

It is a novel concept that the biological reaction rates can be modified by non-enzymatic chemical catalysts thereby drastically changing human processes. Chemical catalysis has been applied to a myriad of inorganic and organic reactions and huge chemical complexes produce billions of dollars of products used in about every endeavor of mankind, however its use in biological processes has been limited. The present invention is related to applications of chemical catalysis and chemical reaction rate processes to dermal effects. It is a new approach to provide favorable treatment for dermal conditions as described in greater detail hereinbelow.

The need continues to exist for improved skin care compositions useful in the treatment of skin disorders, improvement in skin condition and in promotion of hair growth that utilize biologically compatible components to achieve desired dermatological clinical endpoints such as dermal cell repair or follicle stimulation through their biological reactivity.

### OBJECTS OF THE INVENTION

It is an object of the invention to provide improved skin care compositions useful in the treatment of skin, skin disorders and in promotion of hair growth.

It is an additional object of the invention to provide improved compositions and methods useful in the treatment of skin disorders and in promotion of hair growth that utilize cosmetically compatible bio-activating organic catalysts which promote and enhance cellular mechanisms to achieve desired dermatological clinical endpoints.

It is a still further object of the invention to provide improved compositions and methods useful in the treatment of skin disorders and in promotion of hair growth that utilize cosmetically compatible bio-activating organic catalysts to achieve or enhance dermal cell repair and follicle stimulation.

Any one or more of these and/or other objects of the invention may be readily gleaned from a review of the description of the invention which follows.

### SUMMARY OF THE INVENTION

In accordance with the above stated objects, the instant invention provides improved skin care/hair care compositions useful in the treatment of skin, skin disorders and in promotion of hair growth that utilize bio-activating organocatalysts and optional components to achieve dermal cell repair and follicle stimulation. Compositions of the instant invention comprise an effective amount of a bio-activating organocatalyst (as defined in greater detail herein) in combination with carrier, and preferably includes components or ingredients which are typical for skin care or hair care products. In addition, compositions according to the present invention may include effective amounts of novel, including synergistic combinations of exfoliating agents, peroxidant reducing agents, and trace metal catalysts. All of these compositions may be used to provide useful improvements in hair growth, skin improvement (in condition, tone and appearance), to treat wounds, including skin ulcers, skin inflammation, acne, keratoses and for insect bite relief.

In alternative embodiments, compositions according to the instant invention may be used to treat sore throat, stomach ulcers, and cancer, especially including stomach cancer and skin cancer, breast cancer, intestinal cancer, cardiovascular diseases and disease states, including atherosclerosis, an aging heart (by enhancing contraction efficiency, for example, by providing a positive inotropic effect or by enhancing a favorable heart rate by increasing the growth and repair of vital neurological systems operating at an efficiency nature designed), to treat kidney failure and avoid end-stage renal disease, and to treat, rebuild and repair damage to tissues caused by infectious disease, including diseases caused by microbes, including viruses, bacteria and fungi. In these methods, effective amounts of compositions are administered to a patient in need of therapy for an aforementioned disease state or condition.

Compositions according to the present invention comprise an effective amount of bio-activating organocatalyst in combination with a carrier as defined herein, preferably along with additional traditional components which are used in hair care and/or skin care compositions, including for example, emollients such as oils, for example, mineral oil, petrolatum or synthetic oils, surfactants, emulsifiers, conditioning agents, including hair conditioning agents, coloring agents, dyes, pigments, preservatives, sunscreens, UV-absorbing compounds, moisturizing agents, vitamins, minerals, among others, including oil absorbents, antimicrobial agents, binders, buffering agents, denaturants, cosmetic astringents, external analgesics, film formers, humectants, opacifying agents, stiffening agents, perfumes, skin soothing and healing agents, propellants, skin penetration enhancers, solvents, suspending agents, cleansing agents, thickening agents, solubilising agents, waxes, sunscreens, sunless tanning agents, antioxidants and/or radical scavengers, chelating agents, anti-acne agents, anti-inflammatory agents, desquamation agents/exfoliants, organic hydroxy acids and natural extracts, among others, all in effective amounts. These additional components may be added to enhance the composition in promoting or improving hair growth, the condition, tone and appearance of skin, to treat wounds, to rejuvenate skin, to treat skin (including scalp) inflammation, keratoses, acne and for insect bite relief, among other conditions.

In additional embodiments according to the present invention, in addition to a bio-activating organocatalyst and carrier, compositions further optionally comprise effective amounts of one or more of a redox agent or antioxidant/reducing agent, preferably as an enediol-containing component such as an ascorbate derivative or dihydroxy maleic, lipoic acid, dihydrolipoic acid or a derivative, or cholesterol or a derivative, optionally (and preferably) a dermatologically acceptable transition metal-containing component as otherwise disclosed herein such as ferrous histidine, a carrier and optionally, a dermatologically active enzymatic component such as coenzyme CoQ10 (which may be used preferably as a component in certain hair care formulations of the present invention) and optionally a desquamation/exfoliating agent, preferably as a dermatologically acceptable acid or ester. In certain preferred compositions of the instant invention, the redox agent, preferably as an enediol-containing component such as an ascorbate derivative or other redux agent such as dihydroxymaleic acid, lipoic acid, dihydrolipoic acid or cholesterol, undergoes an oxidation reaction with the transition metal-containing component to produce hydrogen peroxide which enhances dermal health and hair growth. This effect may be additive or synergistic with the bio-activating organocatalysts according to the present invention. In alternative embodiments, an effective amount of a topical fever-producing agent and/or chemical irritant can be used in the present compositions in place of (i.e., as a replacement for) or in addition to the redox agent and/or the optional transition metal-containing component.

Compositions of the instant invention optionally contain a dermatologically active acid as a desquamation/exfoliating agent that may be a cosmetically active acid or a pharmaceutically active acid, such as, for example, a hydroxy acid, ascorbic acid or a derivative thereof, lipoic acid, dihydrolipoic acid, or a combination thereof, which also may be included as a redox agent in compositions according to the present invention.

Compositions of the instant invention provide a visible improvement in skin condition shortly following application of the composition to the skin. Such improvement involves a decrease in redness or swelling in dry or inflamed skin, improvements to skin imperfections such as textural discontinuities (including those associated with skin aging, such as age spots and keratoses) and other imperfections, and enhancing skin tone or color. In addition, compositions according to the present invention may be used to improve damaged or irritated skin. Compositions according to the present invention may also be used to promote wound healing or to treat skin inflammation, acne or insect bites. Significantly, application of compositions of the instant invention to the human scalp induce hair growth.

In preferred embodiments, the bio-activating organocatalysts are preferably selected from the group consisting of polyproline (from 100 mer and above, preferably about 100 mer to about 1000 mer), oligoproline (from 2 to about 100 mer, preferably about 2 to 10 mer) proline and its derivatives such as d,l-, d- or l-proline, d- or l-4-hydroxylproline, d or l-proline-t-butyl ester, N-acetyl-l-proline, N-acetyl-d-proline, l-histidine, l-phenylalanine, polyphenylalanine (from 100 mer and above, preferably about 100 mer to about 1000 mer), oligophenylalanine (from 2 to about 100 mer, preferably about 2 to 10 mer), polymeric and oligomeric mixtures (preferably polypeptides or oligopeptides) of proline and phenylalanine (from 2 to more than 1000 mer, preferably 2 to 100 mer, more preferably 2 to 10 mer), imidazolidone and its derivatives, such as 2-imidazolidone-4-carboxylic acid, quinine and its derivatives and salts such as quinine sulfate, quinine hydrosulfate, quinine HCL, quinidine and its salts including quinidine hydrochloride, quinidine sulfate, quinidine gluconate, chloral hydrate, mixtures of proline and aminobutyric acid (1-proline/aminobutyric acid as oligo or polymeric aminoacid multicomponent), mixtures of 1-proline and glycine (1-proline/glycine as oligo or polymeric or aminoacid multicomponent), pyridine derivatives including 4-dimethylaminopyridine, triazole, as well as pharmaceutically acceptable salts or polymorphs of any one or more of the above-described organocatalysts, thereof, where applicable, including enantiomerically pure or enriched materials, as well as racemic mixtures of these compounds. The use of proline or proline derivatives such as l-4-hydroxyproline or is preferred. The term “mer” refers to the number of monomeric units within a given oligopeptide or polypeptide.

In certain preferred embodiments, the invention provides an improved skin care composition which further comprises any one or more of lipoic acid, dihydrolipoic adcid, ascorbyl palmitate, an exfoliant cream base, ferrous histidine and coenzyme CoQ10.

The invention is described further in the following detailed description.

The present invention represents an unexpected result in that bio-activating organocatalysts can influence biological reactions and promote hair growth and skin conditioning and treatment as otherwise described herein in combination with a pharmaceutically acceptable carrier. The inclusion of other components as otherwise described herein, produces even greater effects in combination with the presently described bio-activating organocatalysts. Conventional dermatological sciences counsel for the use of antioxidants as anti-aging agents to avoid free radical formation, whereas certain compositions according to the present invention rely on the formation of controlled free radical reactions that produce peroxide for much of its intended effect of promoting dermal stimulation and hair growth. When combined with the biological activity of bio-activating organocatalysts according to the present invention, the additive or even synergistic result for skin treatment, skin conditioning or hair growth is substantial.

### DETAILED DESCRIPTION OF THE INVENTION

As used herein, the following terms have the following respective meanings. The term “dermatologically-acceptable,” as used herein, means that the compositions or components thereof so described are suitable for use in contact with human skin without undue toxicity, incompatibility, instability, allergic response, and the like.

The term “subject” or “patient refers to any “animal” to whom or to which the compositions according to the present invention may be applied to favorably effect a condition or disease state of the skin or scalp, including the growth of hair. Animals preferably refer to mammals and also to humans, depending upon the skin or scalp condition to be treated or improved within the context of use or treatment.

“Bio-activating organocatalyst” or “organocatalyst” refers to a biologically compatible organic (non-metallic) catalyst which enhances a biological process or reaction in promoting the healing, repairing or conditioning of skin or in growing hair. These compounds are stable in water, are non-toxic and are non-irritating to the skin. While not being limited by way of theory, it is believed that these catalysts activate the dermal pathway to growth and repair. These organic molecules, activate the corium-papilla layer of skin and hence, set into action the living dermal pathway to growth and repair.

Useful organocatalysts can be described by use of computational chemistry. Libraries may be readily constructed from the following classes of compounds: alkaloids, amino acids, ketones, peptides, sulfurylides and derivatives of 4-dimethylaminopyridine, among numerous others, including for example, d,l-, d- or l-proline, d- or l-4-hydroxylproline, d- or l-proline-t-butyl ester, N-acetyl-l-proline, N-acetyl-d-proline, d- or l-histidine, d- or l-phenylalanine, polyphenylalanine (from 100 mer and above, preferably about 100 mer to about 1000 mer), oligophenylalanine (from 2 to about 100 mer, preferably about 2 to 10 mer), polymeric and oligomeric mixtures (preferably polypeptides or oligopeptides) of proline and phenylalanine (from 2 to more than 1000 mer, preferably 2 to 100 mer, more preferably 2 to 10 mer), imidazolidone and its derivatives, such as 2-imidazolidone-4-carboxylic acid, quinine and its derivatives and salts such as quinine sulfate, quinine hydrosulfate, quinine HCL, quinidine and its salts including quinidine hydrochloride, quinidine sulfate, quinidine gluconate, chloral hydrate, polypeptide or oligopeptide mixtures (from 2 to more than 1000 mer, preferably 2 to 100 mer, more preferably 2 to 10 mer) of proline and aminobutyric acid (1-proline/aminobutyric acid as oligo or polymeric aminoacid multicomponent), mixtures of 1-proline and glycine (1-proline/glycine as oligo or polymeric or aminoacid multicomponent), pyridine derivatives including 4-dimethylaminopyridine, triazole, or mixtures thereof, as well as pharmaceutically acceptable salts, of any one or more of the above-described organocatalysts, thereof, where applicable. Enantiomerically pure or enriched compounds as well as racemic mixtures of these compounds may be used in the present compositions. Preferred organocatalysts for use in the present invention include proline or any one or more of its derivatives and imidazolidone, particularly 2-imidazolidone-4-carboxylic acid. Mixtures of organocatalysts may also be used.

In certain aspects of the invention, in particular, where the bio-activating organocatalyst is less soluble in the various components used to make the final composition, the organocatalysts included may be sub-divided into very small particles to enhance activity. In these embodiments, it may be preferred to finely divide the organocatalyst to micron, submicron or nanometer size or even smaller to enhance the activity of the organocatalyst in the composition. Small particle size may increase the activity of the bio-activating organocatalyst in topical applications, i.e., on the skin, hair/scalp or other keratinous tissue.

Bio-activating organocatalysts for use in the present invention are included in compositions in effective amounts, generally ranging from about 0.001 to about 50% by weight or more, about 0.5% to about 35%, about 0.1% to about 25%, preferably about 0.25% to about 20% by weight, preferably about 0.5% to about 15% of a final composition depending upon the application and activity of the bio-activating organocatalysts which includes at least one or more bio-activating organocatalyst and a pharmaceutically acceptable carrier.

The suitability of compounds for use in the present invention may be readily screened to determine the bio-reactivity of compounds in stimulating dermal cells. Those compounds which stimulate dermal cells in the assay are expected to be useful as bio-activating organocatalysts. In this assay, compounds are applied to the dermis of an animal and potentially bio-reactive compounds (i.e., those which might function as bio-activating organocatalysts) produce a multiplicity of very small, pin-head like projections on the epidermis produced by activation of the papilla-corium layer. Papilla have blood vessels and nerves interlaced and they nourish every hair follicle. The activation of the cells of the papilla corium layer ensues for example (preferably) for about 2-4 hours and is from slight to gross depending on the formulation and returns to the normal skin landscape. The bioreactivity of the formula can be judged by the intensity and time of the reaction. All compounds showing bioreactivity may be used in the present invention, with those which are more highly bioreactive being more preferred for more significant effects.

In the case of baldness, the present invention can speed the growth and restoration of hair on the scalp and other areas of the dermis. The rate of reaction in baldness is so low that only extremely fine vellus hair covers the scalp (the k₂ rate in the above described reaction is very, very low). Using bio-activation organocatalysts of the present invention can activate the dermis and scalp (k₂ can be dramatically increased) to produce an abundant hair growth. This represents an enormous increase in reaction rate for the human synthetic production of keratin hair fibers. Additionally, organic synthesis which is catalyzed by bio-activating organocatalysts according to the present invention proceeds at extraordinary speeds to produce cells, cell walls, and functional follicle structures. Thus, the compositions according to the present invention activate papilla in the papilla-corium layer of skin, thus increasing and enhancing new hair growth in animals, including humans.

In the case of skin improvement, e.g. wrinkle reduction, k₂ reactions at the surface of the skin are increased. Penetration of the new catalysts below the surface of the skin (especially where the compositions contain an effective amount of a skin penetration enhancing agent) to reach the papilla immediately speeds up k₂ reactions related to improving skin structure and providing the necessary skin scaffold molecules elastin, fibrin, collagen, etc. along with fats that increase the thickness of the skin and reduce and eliminate fine wrinkles. The activated papilla are seen as slightly swollen during this accelerated chemical reaction rate period. After the catalysts have been exhausted (often, within 2-3 hours), chemical reactions return to normal and beautiful, smooth skin results.

In the case of keratoses and age spots, which occur as a result of skin damage from environmental factors, k₁ reactions (see above) have caused disagreeable structures on the skin. The normal body processes cannot do much about removing this condition because k₂ reactions are too slow to compensate for the enhanced k₁ reactions. However, with a period application of the compositions according to the present invention which include a bio-activating organocatalyst in a carrier, such unsightly structures may be readily removed.

In the case of damaged skin, for example, the treatment of wounds or acne, the desire is to accelerate the body's natural chemical synthesis processes using compositions according to the present invention. In this application, compositions according to the present invention which include effective amounts of at least one bio-activating organocatalyst accelerate the desireable k₂ reaction to enhance wound and skin healing. Because of the multiplicity of the many complex bio-reactions involved in wound and skin healing (as well as hair growth), it is impossible to describe the reactions which are influenced specifically. Suffice it to say that the present compositions are particularly suited for enhancing wound and skin healing, and improving/treating damaged skin. The present invention thus provides simply organocatalysts (some are polymeric/oligomeric in nature, but rely on the activity of individual monomeric units within the polymer or oligomer) which activate and accelerate the very biochemical reactions (in most applications, biosynthesis) which nature is capable of providing. The fact that molecular synthesis is generally stimulated for only brief periods of time (the life of the catalyst is brief, often no more than about 2-3 hours), there is no chance of overdoing the favorable reactions which might produce undesirable results or outcomes. Thus, the compositions of the present invention are particularly suited because of their bioreactivity as well as their safety profile.

“Dermatologically active enzymatic component” includes antioxidant transducers of mitochondrial oxidative phosphorylation such as CoQ₁₀ or H₂CoQ₁₀ (the reduced form of CoQ₁₀). CoQ₁₀ (coenzyme Q10, ubiquinone 50, 2,3-dimethoxy-5-methyl-6-pentacontdacaenyl-benzoquinone) plays a vital role as a rate-limiting carrier for the flow of electrons through the mitochondrial complexes I, II and III of the respiratory chain, thereby maintaining or improving energy (ATP) generation by the mitochondria. It is also a major lipophilic antioxidant. The molecule is located in the inner mitochondrial membrane but is also associated with the membrane of other intracellular organelles. CoQ10 thus maintains redox activity and electron flow across different membranes (Villalba, Crane) and guarantees optimal mitochondrial functioning. The term “H₂CoQ₁₀” refers to the reduced form of CoQ₁₀, otherwise known as ubiquinol. These components are optionally included in compositions according to the present invention, although in certain hair care/hair growth formulations, the inclusion of this component is very highly preferred. Without being limited by way of theory it is believed that the inclusion of CoQ₁₀ or H₂CoQ₁₀ assists in getting hair follicles to begin to produce hair and that the reaction catalysts accelerate the process. Thus, CoQ₁₀ or H₂CoQ₁₀ functions favorably in the present compositions as an initiator or promoter in the hair growth process.

“Redox agents” or “redox agents that produce peroxide” are agents which are optionally included in the present invention to produce hydrogen peroxide and enhance the bioactivity of the present compositions. Exemplary redox agents include ascorbic acid (as well as ascorbate and ascorbate esters and other ascorbate derivatives and salts as described in greater detail herein) and dihydroxy maleic acid (which is preferred and may include esterified forms as well as salts), among other compounds, especially those compounds which contain an enediol moiety, as set forth below.

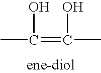

Ascorbic acid and derivatives thereof may be used as redox agents in the present invention as optional agents. Ascorbic acid derivatives suitable for use in the instant invention include, but are not limited to, magnesium ascorbyl phosphate; sodium ascorbyl phosphate; sodium ascorbate; and ascorbyl glucosides. Ascorbic acid and derivatives thereof useful in the invention include, but are not limited to, ascorbyl caprilate, ascorbyl monoate, ascorbyl undeconate, ascorbyl laurate, ascorbyl trideconate, ascorbyl myristate, ascorbyl pentadeconate, ascorbyl palmitate, ascorbyl heptadecanate, ascorbyl stearate, ascorbyl monodecanate, and ascorbyl arachidate. Ascorbic acid and derivatives thereof useful in the invention also include metallic salts of ascorbic acid, including but not limited to sodium, calcium, and magnesium salts.

Preferred enediol-containing components include ascorbate derivatives and salts such as ascorbic acid-2-sulphate dipotassium salt, ascorbic acid-2-phosphate sequimagnesium salt, ascorbic acid-2-polyphosphate sequimagnesium salt and ascorbic acid-2-sulfate-tin. Note that these enediol containing compounds may also serve in certain instances as dermatologically acceptable acids or esters (desquamation/exfoliating agents) in the present compositions and methods. The inclusion of dihydroxymaleic acid or its pharmaceutically acceptable salt forms may be preferred in certain embodiments according to the present invention. In other embodiments, redox agents include lipoic acid, dihydrolipoic acid or its salts or derivatives, cholesterol or its salts or derivatives (especially an ester of cholesterol), vitamin E or its salts or derivatives (especially vitamin E esters), flavanones, flavone, flavanol, gallic acid or its salts or derivatives including esters such as propyl gallate, thymol, quercetin, rutin, hydroxytyrosol, caffeic acid, ellagic acid, cysteine, N-acetyl cysteine, caffeic acid, reduced glutathione, uric acid, 2- or 3-butylated hydroxyanisole (BHA), butylated hydroxytoluene (BHT), tertiary butylated hydroquinone, homeocysteine, trolox (water soluble form of vitamin E), N,N′-Diphenyl-p-phenylene diamine, promethazine, CoQ₁₀ or preferably reduced CoQ₁₀ (H₂CoQ₁₀ or Ubiquinol), allopurinol, probucol, and pharmaceutically acceptable salts and derivatives thereof as well as mixtures of these agents. The term “derivatives” within the context of redox agents refers to pharmaceutically acceptable salts and prodrug forms of these redox agents, such as esters. Note that certain reducing agents which may be included in the present invention (for example, the phenolic compounds, thymol and quercetin) may chelate copper preferentially to or even to the exclusion of any reduction in Cu⁺⁺. Preferred redox agents for use in the present invention include ascorbic acid or its pharmaceutically acceptable salts and derivatives, dihydrolipoic acid (reduced thiooctic acid) or its pharmaceutically acceptable salts and derivatives, cysteine or N-acetyl cysteine or its pharmaceutically acceptable salts and derivatives, or reduced glutathione or its pharmaceutically acceptable salts and derivatives, or mixtures thereof. These reducing agents reduce both CoQ₁₀ as well as copper (Cu⁺⁺).

“Desquamation/exfoliating agents” are agents which are optionally included in compositions according to the present invention and enhance the skin appearance benefits of the present invention. They set in motion in an accelerating way, the skin's exfoliating process. It is a specific area of attack that sets up cell alarm signals in the dermal (including hair) processes to repair damage that is occurring. Hence, anything that affects the outermost layer of the dermis as part of the exfoliating process is contemplated for use in the instant invention. For example, the desquamation agents tend to improve the texture of the skin (e.g., smoothness). A variety of desquamation agents are known in the art and are suitable for use herein, including organic hydroxy acids (including alpha and beta hydroxy acids) such as salicylic acid, glycolic acid, lactic acid, 5-octanoyl salicylic acid, hydroxyoctanoic acid, hydroxycaprylic acid, and lanolin fatty acids. One desquamation system that is suitable for use herein comprises sulphydryl compounds and zwitterionic surfactants. Another desquamation system that is suitable for use herein comprises salicylic acid and zwitterionic surfactants. Additional exfoliating agents include, for example, protease or peptase enzymes (natural and bio-engineered) as well as other polypeptide compositions well-known in the art, bio-mimetic compounds that mimic alpha hydroxyl acids and include peptides, synthetic compounds that cut proteins successfully, and bioactive metals such as manganese, tin and copper (which may be included for its exfoliating properties quite separate from its metal catalysis characteristics), as well as natural soy-based products, such as those in the Johnson & Johnson Aveeno™ product line.

Quite unexpectedly, compositions according to the present invention produce increased hair growth and smooth skin texture, which appears to be facilitated through additional growth and repair mechanisms by increasing the rates of repair and growth reactions through use of the bio-activating organocatalysts and which, in some embodiments which include optional agents, are additionally stimulated by the production of hydrogen peroxide. Thus, it is the increased growth and repair rates of the bio-activating organocatalysts in the first instance, and in certain embodiments which include optional agents, the increased growth and repair rates through catalysis as well as a combination of the exfoliating agents plus hydrogen peroxide signaling of cellular growth and repair mechanisms which represents an important aspect of certain embodiments of the present invention which relate to increased hair growth and skin treatment.

The term “dermatologically acceptable acid or ester” refers to certain optional desquamation/exfoliating agents and includes hydroxy acids such as alpha- or beta-hydroxy acids, poly-hydroxy acids, or any combinations of any of the foregoing. Preferably, the hydroxy acid is an alpha-hydroxy acid. Examples of alpha hydroxy acids include, but are not limited to, glycolic acid, lactic acid, malic acid, tartaric acid, pyruvic acid, citric acid, or any combination of any of the foregoing. Alpha hydroxy acids are preferred in certain compositions for their ability to stimulate dermal cells to produce collagen and fibrinogen. Beta-hydroxy acids include, but are not limited to, salicylic acid. Lipoic acid and dihydrolipoic acid may also be used as dermatologically acceptable acids or esters.

A “dermatologically acceptable transition metal-containing component”, an optional component of the present compositions, includes compositions containing copper, iron, cobalt, manganese or tin such as copper histidine, iron (ferrous) histidine, ferrous EDTA, and copper EDTA and iron (ferrous) desferrioxamine and can include other salts such as the chloride, sulfate, (e.g. ferrous ammonium sulfate), nitrate and lactate salts of these metals, among others, including chelated complexes, as described below. Transition metals that are particularly preferred include Cu⁺², Cu⁺, Fe⁺², Fe⁺³, and Co⁺², as discussed above, preferably as chelates. Without intending to be bound to or limited by any theory, it is believed that transition metals are a key element in promoting beneficial controlled free radical production in certain formulations of the instant invention which include these components. The reaction of ascorbate derivatives with transition metals favors beneficial hydrogen peroxide production and is synergistic in producing favorable effects in combination with the bio-activating organocatalysts used in the present compositions.

In certain embodiments of the instant invention, the dermatologically acceptable transition metal-containing component is complexed with chelating agents such as EDTA, lactate, desferrioxamine, ethylene diammonium sulfate and tripeptide (diglycyl-1-histidine). Another set of chelates which may be used in the present invention are ferrous O-trensox and and ferric O-trensox, which are hydroxyquinoline-based iron chelators, which do not catalyze so-called Fenton reactions which produce biologically damaging hydroxyl radicals. See, J. Am. Chem. Soc., 117, 9760 (1995). The use of iron EDTA represents a preferred embodiment. When used, such chelating agent complexes provide beneficial reaction control in free radical production. Iron chelators, especially iron EDTA or iron desferrioxamine, are preferred for use in the present invention. Selective preferred chelators covering a range of ionization constants or having affinity constants ranging from about 10⁵ up to about 10⁵³ (even more preferably up to 10⁴³, within this range), are particularly useful for inclusion in the present invention.

In metal chelate-ascorbate systems, the histidine metal chelate causes only limited damage to DNA while EDTA chelates do not catalyze the Fenton reaction that can produce damaging .OH radicals. It is believed that in these tight chelates there is insufficient metal ion available to decompose H₂O₂. No Fenton reaction is therefore possible and DNA scission is quenched. While less tightly bound transition metals with ascorbate are known to cause attack on DNA in vitro, recent work on the new biology of ascorbic acid demonstrates that in vivo systems containing ascorbate and transition metals, DNA is not attacked, as ascorbate acts as a protective agent.

Affinity Constants of Metal Chelates

| Chelate | Affinity Constant |
|---|---|
| Copper lacate | 10¹⁰-10¹² |
| Copper histidine | 10¹⁶ |
| Copper EDTA | 10²³ |
| Copper gluconate | 10⁶ |
| Ferrous lactate | 10¹² |
| Ferrous histidine | 10¹⁶ |
| Ferrous EDTA | 10²⁵ |
| Desferrioxamine | 10³⁰ |
| Ferric EDTA | 10²⁵ |
| Glycolic acid | 10⁵ |
| Copper tripeptide (GHG) | 10¹⁶ |
| Ferric salicylic acid | 10¹⁶ |
| Ferric catechol | 10²⁰ |

From the above, it is seen that the optional inclusion of selective catalytic metal activity can produce a variety of therapeutically beneficial results. All that is required is that the metal ion chelate have special arrangements that provide either full or limited access to O₂ and H₂O₂ and further that the ionization (or affinity) constant of the chelate be sufficient to control the specific end products of the reaction. Thus, the scope of the present invention includes a broad range (scope) of metal chelators to achieve various effects in dermal treatment.

In the present invention, “dermatologically acceptable chemical irritants” are optional components for use in the present invention. These agents also may be used as alternatives to redox agents and transition metal containing components in the present invention. These are agents which produce a measured and non-damaging skin irritation, at least a general reddening of the skin which is exposed to the agent and in certain instances, swelling and related physiological responses. These agents are known to produce a dynamic complex of cytologic and histologic reactions which occur in the affected blood vessels and tissues being exposed to these agents. The skin to which these agents are applied typically respond to these agents in local reactions and morphological changes, destruction or removal of the irritant from the tissue, and responses which lead in the tissue to repair or healing. The irritation which occurs from these agents and the physiological response to that irritation is advantageously used in the present invention. These agents may be used in addition to, or instead of (i.e., as replacements for) redox agents/transition metal-containing components in the present invention. Examples of such agents include various proteolytical enzymes as well as other enzymes, alcohol, including grain spirits or rubbing alcohol (isopropanol), ammonia spirit, aromatics, creosote, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh, and topical mild fever agents as described below, among others.

“Topical mild fever agents” are those agents which fall under the rubric of dermatologically acceptable chemical irritants and induce a very mild topical local fever in skin which may be used to further promote stimulation of skin and/or hair follicles in the present invention. Although not considered exfoliating agents, these agents are similar to exfoliating agents in that they stimulate the skin for further growth, often, however, without attacking cells (in the dermal layer) from the skin. These agents produce mild elevation of skin temperatures and pyrogens. These agents include, for example, capsaicin, piperine, mustard, nicotinic acid, camphor, menthol and limonene, among other agents or irritants. These agents may be used in addition to, or instead of (i.e., as replacements for) redox agents and transition metal containing components.

The term “wound” means a superficial or topical wound of the skin such as a burn, cut, scrape, scratch, minor irritation or surgical wound, as well as scars which occur secondary to wounds. The term inflammation means inflammation of the skin, whether that irritation is considered a wound or is simply considered damaged skin. “Damaged skin” is skin which has been sunburned, contains dermal lesions, irritation or imperfections which do not rise to the level of a wound and can include wrinkles and other conditions which exist as a consequence of natural processes, including aging, exposure to the sun, etc.

The term “skin smoothness” is used to refer to tactile skin properties that encompass one or more of the following: roughness, suppleness, elasticity, softness, friction, dryness, scaling, and pliability. In certain embodiments, compositions according to the present invention enhance the smoothness of skin, including damaged skin.

The term “acne” is used to describe an inflammatory follicular, popular and pustular eruption involving the pilo sebaceous apparatus, in all of its many forms (generalis, albida, artificialis, conglobata, erythmatosa (rosacea), fulminans, vulgaris, etc.), as traditionally described and understood by those of ordinary skill as well as well as scars which result from acne. The term “keratosis” or “keratiasis” is used to describe any lesion on the epidermis marked by circumscribed overgrowths of the horny layer, in all of its many forms (actinic, follicularis, etc.).

“Carriers” include compositions suitable for topical application to the skin (including the scalp) or related keratinous tissue within which the essential materials and optional other materials are incorporated to enable the essential materials and optional components to be delivered to the skin or related keratinous tissue at an appropriate concentration. The carrier can thus act as a diluent, dispersant, solvent, or the like for the various components of the instant compositions including particulate material(s) and the actives which ensure that they can be applied to and distributed evenly over the selected target at an appropriate concentration.

The carrier can be solid, semi-solid or liquid. Highly preferred carriers are liquid or semi-solid, such as creams, lotions and gels. Preferably, the carrier is in the form of a lotion, cream or a gel, more preferably one which has a sufficient thickness or yield point to prevent the particles from sedimenting. The carrier can itself be inert or it can possess dermatological benefits of its own. The carrier should also be physically and, chemically compatible with the essential components described herein, and should not unduly impair stability, efficacy or other use benefits associated with the compositions of the present invention. Preferably, active components are micronized for inclusion into the compositions for enhanced activity.

The type of carrier utilized in the present invention depends on the type of product form desired for the composition. The topical compositions useful in the subject invention may be made into a wide variety of product forms such as are known in the art. These include, but are not limited to, lotions, creams, gels, sticks, sprays, ointments, pastes, and mousses. These product forms may comprise several types of carriers including, but not limited to, solutions, aerosols, emulsions, gels, solids, and liposomes. Preferred carriers contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), polypropylene glycol (e.g. of MW 425-2025), glycerol, butylene glycol, 1,2,4-butanetriol, sorbitol esters, 1,2,6-hexanetriol, ethanol, iso-propanol, sorbitol esters, ethoxylated ethers, propoxylated ethers and combinations thereof. The diluent is preferably liquid. Water is an especially preferred diluent. The composition preferably comprises at least about 60% of the hydrophilic diluent, although significantly lower amounts may be used, depending upon the final formulation.

Preferred carriers comprise an emulsion comprising a hydrophilic phase, especially an aqueous phase, and a hydrophobic phase e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic oil phase and from about 1% to about 99% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. Preferred compositions herein may be oil-in-water or water-in-oil emulsions.

“Therapeutically effective amount” as used herein means an amount of a composition of the instant invention that, when applied to the skin, including the scalp, of a mammal, produces an intended biological/therapeutic effect, which effect may include moisturizing the skin, reducing irritation, enhancing skin tone, reducing wrinkles, reducing scaling, inhibiting or otherwise treating inflammatory disorders including psoriasis, stimulating skin cell growth, stimulating hair follicles or hair growth, etc.

The term “effective amount” subsumes the term therapeutically effective amount within it and is directed to an amount of a composition, compound or component which produces an intended effect within the context of its use, whether that use is for the improvement in skin, hair growth, treatment of wounds, treatment of inflammation and other skin conditions including acne, keratosis, wrinkles, etc., acne and insect bite relief, or a completely different use within the context of a component's inclusion into a composition according to the present invention. Of course, the final use of the composition may affect the amount of agent to be included within a particular formulation or composition. For example, skin treatment formulations, in contrast to hair growth formulations, may have reduced amounts of certain components which would grow hair, in order to reduce or avoid the growth of hair where such result would not be favored, would have reduced amounts of CoQ10 or other enzymatic active component, or in some cases, even eliminate this component. Wound healing formulations might emphasize the inclusion of an optional redox agent along with the transition-metal containing component. In addition, in the case of acne and/or keratosis treatment, the compositions may exclude organocatalyst therefrom, provided that the redox agent (reducing agent or antioxidant) is included in the formulation along with the transition-metal containing component in effective amounts to treat the condition for which the composition was formulated. Preferably, organocatalyst is included in these formulations in effective amounts.

Note that hair care formulations may be formulated to increase follicle cell growth (increase the size and activity of hair follicles) without producing irritation of the surrounding skin. The formula according to the present invention which are bioreactive in a skin test show a multiplicity of very small, pin-head like projections on the epidermis produced by activation of the papilla-corium layer. Papilla have blood vessels and nerves interlaced. They nourish every hair follicle. The activation ensues for about 2-4 hours and is from slight to gross depending on the formulation and returns to the normal skin landscape. The bioreactivity of the formula can be judged by the intensity and time of the reaction. The above shows that it is possible to have skin bioreactivity by a formulation that does not cause free radical damage to DNA. This test may be used to test or screen for activity of bio-activating organocatalysts according to the present invention.

Note that an alternative formula which may be preferred may optionally include Eucerin™ 9.6 g, ascorbate solution (15% solution of the sodium salt) of 0.2 cc and 0.2 cc of a 1.0% by weight ferrous EDTA solution, as well as an effective amount of a bio-activating organocatalyst, preferably proline or one of its derivatives, or 2-imidazolidone-4-carboxylic acid.

The following Table 1 provides a general overview of individual bio-activating organocatalysts and their general reactivity in the bioreactive skin test as detailed in example 1 of and as otherwise described in the present application.

TABLE 1

| | Concentration | |
|---|---|---|
| Compound | (% by weight) | Bioreactivity |
| d,1-proline | 25 | *** |
| d,1-proline | 10 | ** |
| 1-proline | 25 | *** |
| 1-proline | 2 | * |
| 1-4-hydroxyproline | 25 | *** |
| 1-4-hydroxyproline | 10 | ** |
| 1-4-hydroxyproline | 2 | * |
| 1-proline-t-butyl ester | 20 | *** |
| 1-proline methyl ester HCL | 25 | ** |
| N-acetyl-1-proline | 20 | * |
| 1-phenylalanine | 20 | * |
| 2-imidazolidone-4-carboxylic acid | 20 | *** |
| 2-imidazolidone-4-carboxylic acid | 10 | ** |
| 2-imidazolidone-4-carboxylic acid | 2 | * |
| triazole | 10 | * |
| quinine hydrosulfate | 10 | ** |
| quinine hydrosulfate | 2 | * |
| quinine hydrochloride | 10 | ** |
| quinidine | 2 | * |
| quinidine hydrochloride | 2 | * |
| quinidine sulfate | 10 | ** |
| quinidine gluconate salt | 10 | ** |
| chloral hydrate | 10 | ** |
| 1-proline/aminobutyric acid | 20 | *** |
| 1-proline-glycine octa peptide | 2 | * |
| 4-dimethylaminopyridine | 10 | ** |
| 1-histidine | 10 | ** |

Table guide:
* = slight bioreactivity
*** = gross bioreactivity

The following table 2 provides a general overview of individual optional components and their preferred weight ranges in certain embodiment compositions according to the present invention. Note that other than a bio-activating organocatalyst, the remaining components are optional as otherwise disclosed herein. One of ordinary skill in the art may readily modify the type and amount of components as otherwise taught herein in practicing the present invention.

TABLE 2
Recommended Concentrations-Weight % of Individual Optional
Components- to be added to Bio-activating Organocatalyst

| | Wound | Skin | Hair |
|---|---|---|---|
| CoQ10 | | | |
| range | 0.5-15 | 0-2 | 0.1-10 |
| preferred | 1-3 | 0.1-0.5 | 0.5-1.5 |
| optimum | 2 | 0.3 | 2 |
| Redox Agent | | | |
| range | 0.5-10 | same | same |
| preferred | 0.5-2 | same | same |
| optimum | 2.5 | same | same |
| Metal Salt | | | |
| range | 0.001-5 | 0.001-5 | 0.001-5 |
| preferred | 0.01-1 | 0.01-0.3 | 0.01-0.3 |
| optimum | 0.5 | 0.05 | 0.05 |
| Exfoliating Agent | | | |
| range | 0-15 | 0.5-15 | 0.1-15 |
| preferred | 3-12 | same | same |
| optimum | 10 | same | same |

| | |
|---|---|
| Skin Irritant | Broad range of amount from micro to macro |
| | depending on particular Agent and activity. The range |
| | is extremely large and preferably is |
| | from about 0.001% to about 10%. |

The term “exfoliant cream base” refers to a cream base or lotion which comprises on a weight/weight basis about 1-2% to about 20% (preferably, about 5% to about 15%, more preferably about 10%) by weight of a desquamation/exfoliating agent, preferably an alpha hydroxy acid, more preferably glycolic, lactic acid or a dermatologically acceptable salt; about 2% to about 20% by weight of a plasticizing agent, preferably urea, in a preferred amount ranging from about 5% to about 15%, more preferably about 10% and a standard topical cosmetic/pharmaceutical lotion or cream base making up about 60% to about 96%, more preferably, about 65% to about 93%, even more preferably about 80% of the exfoliant cream base.

The topical compositions of the present invention may comprise a wide variety of additional optional components, provided that such additional optional components are physically and chemically compatible with the essential components described herein, and do not unduly impair stability, efficacy or other use benefits associated with the compositions of the present invention. These optional components may be dispersed, dissolved or the like in the carrier of the present compositions. These optional components include emollients, oil absorbents, antimicrobial agents, binders, buffering agents, denaturants, cosmetic astringents, external analgesics, film formers, humectants, opacifying agents, perfumes, pigments, skin soothing and healing agents, preservatives, propellants, skin penetration enhancers, solvents, suspending agents, emulsifiers, cleansing agents, thickening agents, solubilising agents, waxes, sunscreens, sunless tanning agents, antioxidants and/or radical scavengers, chelating agents, anti-acne agents, anti-inflammatory agents, desquamation agents/exfoliants, organic hydroxy acids, vitamins and natural extracts. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993) can also be used in the present invention.

Another optional component, which may be preferred as an antioxidant for use in the present invention is alpha lipoic acid, or its pharmaceutically acceptable salt form, preferably in its reduced form, which may be included as a defoliation/desquamation agent or separately, for its beneficial characteristics as an antioxidant. This component may be added in amounts ranging from about 0.005% to about 10.0%, more preferably about 0.01% to about 1% by weight (when used as an antioxidant as opposed to its alternative use as an exfoliating/desquamation agent) for its beneficial antioxidant effects on cells, which may provide benefit for the cellular growth and repair mechanisms which are facilitated by compounds according to the present invention.

A safe and effective amount of a desquamation/exfoliating agent may be added to the compositions of the subject invention, more preferably from about 0.1% to about 20%, even more preferably from about 0.2% to about 10%, also preferably from about 0.5% to about 4% of the composition. In addition, agents which induce a very mild topical local fever in skin (“topical mild skin agitants”) such as pepper (capsaicin), piperine, mustard, nicotinic acid, camphor, menthol and limonne among others, may also be used in place of, or in addition to, the desquamation agent or exfoliant, essentially the same amount as the desquamation/exfoliating agent.

Compositions according to the present invention may also include a peptide such as a tripeptide, alone or in combination with a metal such as a copper (II) or tin (II) chelate of the tripeptide Gly-L-His-L-Lys and other peptides or additives which are known to enhance wound healing and to otherwise improve attributes of skin.

The pH range of compositions of the instant invention is approximately 4-9, more preferably 5-7, and even more preferably about 6-7.

The invention is described further in the following examples, which are illustrative and not limiting. All percentages, parts and ratios are by weight of the total composition, unless otherwise specified. All such weights as they pertain to listed ingredients are based on the specific ingredient level and, therefore, do not include solvents, carriers, by-products, filler or other minor ingredients that may be included in commercially available materials, unless otherwise specified.

### BIOREACTIVITY TESTING AND EXAMPLES

In order to test the relative bioreactivity of organocatalysts according to the present invention, a number of potential organocatalysts were formulated into a skin cream using Eucerin™ at concentrations ranging from 2% to 25% by weight of the final skin cream formulation. See table 1A below. These skin creams were formulated by dissolving the organocatalyst at a desired amount into the Eucerin. The formulated compositions were then tested for skin bioreactivity according to the method previously described hereinabove.

Essentially, the skin cream formulations containing organocatalyst are tested on skin. Those compositions which are bioreactive in the skin test show a multiplicity of very small, pin-head like projections on the epidermis produced by activation of the papilla-corium layer. The activation continues for about 2-4 hours and is from slight to gross depending on the formulation and returns to the normal skin landscape. The bioreactivity of the formula can be judged by the intensity and time of the reaction. The results of the tested formulations are presented in Table 1A below. Note that the four non-organocatalysts at the bottom of the table all were completely unreactive in the test system, as expected.

TABLE 1A

| | Concentration | | |
|---|---|---|---|
| Compound | (% by weight) | Bioreactivity | Comment |
| d,1-proline | 25 | *** | |
| d,1-proline | 10 | ** | |
| 1-proline | 25 | *** | |
| 1-proline | 2 | * | |
| 1-4-hydroxyproline | 25 | *** | |
| 1-4-hydroxyproline | 10 | ** | |
| 1-4-hydroxyproline | 2 | * | |
| 1-proline-t-butyl ester | 20 | *** | |
| 1-proline methyl ester HCL | 25 | ** | |
| N-acetyl-1-proline | 20 | * | low solubility |
| 1-phenylalanine | 20 | * | low solubility |
| 2-imidazolidone-4-carboxylic acid | 20 | *** | |
| 2-imidazolidone-4-carboxylic acid | 10 | ** | |
| 2-imidazolidone-4-carboxylic acid | 2 | * | |
| triazole | 10 | * | |
| quinine hydrosulfate | 10 | ** | |
| quinine hydrosulfate | 2 | * | |
| quinine hydrochloride | 10 | ** | |
| quinidine | 2 | * | |
| quinidine hydrochloride | 2 | * | |
| quinidine sulfate | 10 | ** | |
| quinidine gluconate salt | 10 | ** | |
| chloral hydrate | 10 | ** | |
| 1-proline/aminobutyric acid | 20 | *** | |
| 1-proline-glycine octa peptide | 2 | * | |
| 4-dimethylaminopyridine | 10 | ** | |
| 1-histidine | 10 | ** | |
| Eucerin renewal | 100 | no reactivity | |
| disodium tartrate | 20 | no reactivity | |
| propylene glycol | 25 | no reactivity | |
| glucose | 20 | no reactivity | |
| minoxidil | 5 | ± (very slight) | |

Table guide:
* = slight bioreactivity
*** = gross bioreactivity

### ADDITIONAL EXAMPLES

The following examples represent compositions which include additional or optional components which may be added to effective amounts of one or more of the bio-activating organocatalysts according to the present invention. All components' weight percentages may be adjusted to accommodate effective amounts of the organocatalysts according to the present invention, although it is preferred that the inert components be adjusted to accommodate the bio-activating organocatalyst. Amounts of the catalysts in the present compositions may range from 0.001% up to about 50% by weight of the final composition.

### EXAMPLE

Hair Cream

A hair cream of the instant invention is prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that the cream base be adjusted to accommodate the catalyst. The illustrated hair cream can be formulated at room temperature and atmospheric pressure and the resulting reactions are readily controlled to yield the desired composition.

| Component | Weight Percentage |
|---|---|
| *CoQ10 (submicron) | 2 |
| Lipoic Acid (micronized) | 3 |
| Ascorbyl palmitate | 3 |
| **Exfoliant cream base | 91.5 |
| Ferrous histidine | 0.5 |

*Bio-Q ™ Essentials- From Julian Whitaker, Healthy Directions - in 70% by weight soybean oil (5% of composition weight is soybean oil and 2% of composition weight is CoQ10).
**Exfoliant cream base is made of (w/w) 10% by weight lactic acid partial salt, 10% by weight urea and 80% of a standard topical cosmetic/pharmaceutical lotion or cream.

The hair cream of this example may be applied to the skin of a mammal for enhancement of hair growth. Application of the skin cream to the scalp of a mammal in a concentration of between about 0.3 to 0.5 gm/cm² twice daily, may result in hair growth stimulation over a period of about 3 to 4 months.

### EXAMPLE

Gel

A gel of the instant invention is prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that the inert gel base be adjusted to accommodate the organocatalyst. The illustrated gel can be formulated at room temperature and atmospheric pressure and the resulting reactions are readily controlled to yield the desired composition.

| Component | Weight Percentage |
|---|---|
| CoQ10 (micronized) | 2 |
| Lipoic Acid (micronized) | 3 |
| Ascorbyl palmitate | 3 |
| Exfoliant gel base | 88.5 |
| Ferrous histidine | 0.5 |
| Soybean oil | 3 |

The gel of this example may be applied to the skin of a mammal for enhancement of skin health and condition and pH changes and skin tone and color may be monitored. Application of the gel to the scalp of a mammal in a concentration of between about 0.3 to 0.5 gm/cm² twice daily may result in hair growth stimulation over a period of 3 to 4 months.

### EXAMPLE

Lotion

A lotion of the instant invention is prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that inert components in the lotion base be adjusted to accommodate the organocatalyst. The illustrated lotion can be formulated at room temperature and atmospheric pressure and the resulting reactions are readily controlled to yield the desired composition.

| Component | Weight Percentage |
|---|---|
| CoQ10 (Solubilized)* | 2 |
| Lipoic Acid (micronized) | 3 |
| Ascorbyl palmitate | 3 |
| Exfoliant lotion base | 91.5 |
| Copper histidine | 0.5 |

*Q-Gel ™ 100 From Tishcon, Corp., Westbury, New York.

The lotion of this example may be applied to the skin for enhancement of skin health and condition and pH changes and skin tone and color may be monitored. Application of the lotion to the scalp in a concentration of between about 0.3 to 0.5 gm/cm² twice daily may result in hair growth stimulation over a period of 3 to 4 months.

### EXAMPLE

Ointment

An ointment of the instant invention is prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that inert components in the ointment base be adjusted to accommodate the organocatalyst. The illustrated ointment can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Ascorbyl palmitate | 3 |
| Exfoliant ointment base | 91.5 |
| Copper histidine | 0.5 |

The ointment of this example may be applied to the skin of a mammal for enhancement of skin health and condition and pH changes and skin tone and color may be monitored. Application of the lotion to the scalp of a mammal in a concentration of between about 0.3 to 0.5 gm/cm² twice daily may result in hair growth stimulation over a period of 3 to 4 months.

### EXAMPLE

Skin Cream

A skin cream of the instant invention is prepared by mixing the following components in the designated weight percentages through dermatological formulation techniques that are well-known in the art, which may be adjusted to accommodate effective amounts of one or more bio-activating organocatalysts according to the present invention. It is preferred that inert components in the cream base be adjusted to accommodate the organocatalyst. The illustrated skin cream can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Dihydroxymaleic acid | 1 |
| Exfoliant cream base | 91.5 |
| Ferrous histidine | 0.5 |

The skin cream of this example may be applied to the skin of a mammal for enhancement of skin health and condition and pH changes and skin tone and color may be monitored. Application of the skin cream to the scalp of a mammal in a concentration of between about 0.3 to 0.5 gm/cm² twice daily may result in hair growth stimulation over a period of 3 to 4 months.

### EXAMPLE

Skin Cream

A skin cream of the instant invention is prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that inert components in the cream base be adjusted to accommodate the organocatalyst. The illustrated skin cream can be formulated at room temperature and atmospheric pressure and the resulting reactions are readily controlled to yield the desired composition.

| Component | Weight Percentage |
|---|---|
| CoQ10 | 2 |
| Lipoic Acid (micronized) | 3 |
| Ascorbyl palmitate | 3 |
| Exfoliant cream base | 91.5 |
| Copper EDTA | 0.5 |

The skin cream of this example may be applied to the skin of a mammal for enhancement of skin health and condition and pH changes and skin tone and color may be monitored. Application of the skin cream to the scalp of a mammal in a concentration of between about 0.3 to 0.5 gm/cm² twice daily may result in hair growth stimulation over a period of 3 to 4 months.

### EXAMPLE

Skin Creams

Skin creams of the instant invention are prepared by mixing the following components in the designated weight percentages with an appropriate carrier through dermatological formulation techniques that are well-known in the art, which may be adjusted to accommodate effective amounts of one or more bio-activating organocatalysts according to the present invention. It is preferred that inert components in the cream base be adjusted to accommodate the organocatalyst. The illustrated skin creams can be formulated at room temperature and atmospheric pressure and the resulting reactions are readily controlled to yield the desired composition.

Composition A

| Component | Weight Percentage |
|---|---|
| CoQ10 | 2 |
| Lipoic Acid (micronized) | 3 |
| Ascorbyl palmitate | 3 |
| Exfoliant cream base | 91.5 |
| Ferrous EDTA | 0.5 |

Composition B

| Component | Weight Percentage |
|---|---|
| CoQ10 | 2 |
| Lipoic Acid (micronized) | 3 |
| Ascorbyl palmitate | 3 |
| Exfoliant cream base | 91.9 |
| Ferrous ethylenediammonium sulfate | 0.1 |

The skin cream(s) of this example may be applied to the skin of a mammal for enhancement of skin health and condition and pH changes and skin tone and color may be monitored. Application of the skin cream to the scalp of a mammal in a concentration of between about 0.3 to 0.5 gm/cm² may result in hair growth stimulation over a period of 3 to 4 months.

### EXAMPLE

A skin care preparation was made by incorporating the following:

| | |
|---|---|
| CoQ₁₀ | 2% |
| Lipoic Acid | 3% |
| Ascorbyl palmitate | 5% |

into a skin cream. The mixture was transferred to aluminum metal tubes for dispensing. This mixture was shown to grow hair in modest amounts. However, when this formulation was used at the rate of six (6) times per day, astoundingly, it caused hair to fall out. From this experiment, we concluded that there was some growth promoter in the formulation that caused hair growth if used in reasonable amounts.

### EXAMPLE

This formulation contained the dihydro or reduced form of Co Q₁₀ (uibiquinol) as follows:

| | |
|---|---|
| Reduced Co Q₁₀ (Ubiquinol) | 2% |
| Lipoic Acid | 3% |
| Ascorbyl palmitate | 5% |

The skin cream lotion used in the formulation containing the above ingredients contained an exfoliating agent (lactic acid). This combination was transferred to aluminum dispensing tubes.

This composition was beginning to show some activity after only one month and after 3 months it was considered to be a most remarkable hair grower. We concluded that the high bioavailability of Co Q₁₀ in the reduced form (water soluble) was responsible for the improved results. Also, the exfoliating mechanism, induced by the hydroxy acid (lactic acid), was a contributing factor. This composition may be readily modified to accommodate bio-activating organocatalysts of the prsent invention.

A fresh formulation of the composition, above, was put into a glass container (1) and aluminum metal tubes (2). Sample 2 was very substantially better. It was concluded that the aluminum dispensing tube played a definite part in the successful hair growth program—a quite unexpected result.

The two formulations above were put under observation. It was observed that on standing, pressure and odor developed in sample (2). More samples were made from this type of formulation using varied exfoliating creams. The gas was identified as hydrogen (H₂) and the more acidic the formulation the more gas was produced until one acted like a geyser. It was concluded that lactic acid attacked the aluminum tubes and the vigor depended on acidity. It was also concluded that the metals of that alloy are important and appear to be a significant factor in the hair growth exhibited. It was therefore concluded that trace metal catalysts are an important component of the present invention.

### EXAMPLE

The purpose of this example is to demonstrate the use of dihydroxy-maleic acid as an enediol compound. (extension of ascorbic acid as part of a generic class).
The dihydroxy maleic acid DHM was neutralized with NaOH to pH 6.5 and then prepared to a 15% solution.
   Eucerin™ 7.9 g.
   DHM (15%) 1.1 c.c.
   Fe EDTA (1%) 1.0 c.c.
The catalyst Fe EDTA was prepared as a 1% solution of Mohr's salt Fe(NH)₄SO₄.6H₂O) and a molar amount of ethylene diamine tetra acetic acid or Fe EDTA.2Na. Then 10% extra EDTA was added to the final catalyst solution.
   Results
The formulation produced excellent bioactivity on my skin test. No hair growth tests were made as animal studies are necessary. This composition does not cause DNA scission.

### EXAMPLE

Purpose
a) To use Atrac-Tain*as a vehicle.

b) To use sodium ascorbate in place of ascorbyl palmitate

| | | |
|---|---|---|
| Atrac-Tain | 8.0 | g. |
| Soybean oil | 1.1. | g. |
| Sodium ascorbate (1.5%) | 1.0 | cc. |
| CoQ₁₀ (micronized) | 0.2 | g. |
| Copper lactate (0.5%) | 1.0 | cc. |

*contains alpha hydroxy acid

The copper lactate solution was prepared by adding 0.5% copper sulfate pentahydrate to a molar quantity of lactic acid and then pH adjusted to 6.3 with NH₄OH.
   Results
The homogenized mix with soybean oil as a carrier for CoQ₁₀ was very stable.
It diffused into the skin on application as the CoQ₁₀ orange color disappeared.
   It produced an excellent bio-reactive effect in my new skin test.
   The sodium ascorbate successfully replaced ascorbyl palmitate.
   The formulation is similar to the one used to grow hair on animals.

### EXAMPLE

Formulation (2) was diluted with Atrac-tain in a 1 to 4 ratio.

Results
The color of the above formulation is excellent and it is still bioactive. Cu lactate is an excellent chelate form for bioactivity.

### EXAMPLE

Purpose
To use copper histidine as a catalyst with sodium ascorbate.

There is no CoQ₁₀ in the formulation.

| | |
|---|---|
| Eucerin ™ | 9.72 |
| Copper Histidine* | 0.03 cc |
| 1% | |
| Ascorbate (1.5%) | 0.25 cc. |

One drop of saturated Histidine solution is added to the formulation.

Results
Color of mix very good and bioactivity on skin was demonstrated.
This example depends on the copper reaction catalyst for it's effect on skin.
*The metal catalyst Cu Histidine was prepared by adding 1% Copper Sulfate penta-hydrate to a molar quantity of Histidine and then adjusting the pH to 6.3. The salt is Cu Histidine.

### EXAMPLE

| | |
|---|---|
| Atrac-Tain | 4.8 |
| Ascorbate 1.5% | 0.1 c.c. |
| Fe EDTA (1%) | 0.1 c.c. |

Results
Long Term color evidenced on spot plate. Outstanding bioreactivity is very satisfactory on skin.
Note: Another metal-ascorbate reaction catalyst only system.

### EXAMPLE

| | |
|---|---|
| Aveeno (J& J Cream) | 4.8 |
| Ascorbate (15%) | 0.1 cc |
| Fe EDTA (1%) | 0.1 cc |

Add one drop of EDTA saturated solution and allow to equilibrate for 2 days.

Results
Initial color excellent, but on aging, it was not as good. If higher concentrations of ascorbate-iron EDTA catlyst are used the Aveeno mix, on aging, turns dark brown or even black.
Note: There is no alpha hydroxy acid in the formula—just iron-ascorbate reaction catalyst, yet bioactivity was quite good.

### EXAMPLE

Dilute the Aveeno formulation in example (18) in a 1-1 ratio with Aveeno Cream.

### Results

Very definite action, might be sufficient for commercial use even at this high dilution. (1-10×).
Only ascorbate-iron catalyst at work and free radical action is below DNA scission for this reaction.
   In Summary.
   Following was shown by one or more of the examples:
1. Sodium ascorbate can be used as a substitute for ascorbyl palmitate.
2. Dihydroxymaleic acid in place of ascorbate is another example of the enediol generic class.
3. Iron and Copper were chelated so tightly that DNA scission no longer occurs still shows bioreactivity on skin when combined with ascorbate (or an enediol). Iron is definitely the more active catalyst metal.
4. CoQ₁₀ and Alpha hydroxy acids are not necessary for activation of the bioreactivity measurement on skin. Hair growth is unknown.
5. Soybean oil is an excellent transporter of CoQ₁₀ across the skin barrier.
6. J & J Aveeno, Daily Moisturizing Lotion, is an acceptable vehicle for our ascorbate metal reaction catalyst, provided the catalyst is used in the more dilute range.
7. Tightly bound Copper Histidine is an acceptable metal catalyst for the ascorbate metal reaction catalyst. The chelated but somewhat less tightly bound copper in copper lactate is a preferred catalyst based on our skin studies as well as hair growth in humans and animals.

### EXAMPLE

The following composition was prepared and tested in laboratory test animals:

| | | |
|---|---|---|
| Eucerin ™ Renewal* | 33.00 | g |
| Ascorbyl Palmitate | 1.60 | g |
| Copper Lacate | 1.5 | cc of a 1% solution |
| Coenzyme Q | 0.6 | g |
| Soybean Oil | 1.15 | |

*Eucerin ™ Renewal from Beiersdorf, Inc. Wilton, Connecticut, USA.

Copper lactate is 1% salt solution, copper sulfate penta hydrate with a molar quantity of lactic acid.

The ingredients were thoroughly hand mixed for each material added followed by successive intermittent homogenization at high speed being careful not to overheat the emulsion. The ascorbyl palmitate was successfully dispersed for good chemical reactivity so that when the trace metal catalyst (copper lactate) was added in the final step of the formulation procedure, the CoQ₁₀ spontaneously reduced to mostly H₂CoQ₁₀.

The hair growth composition as prepared above was evaluated in the C3H mouse model of hair growth, in parallel with the benchmark 2% Minoxidil. 6-week old female C3H mice were purchased from Taconic Labs and acclimated for 1 week. When all mice were confirmed to be in telogen, approximately 1.5×5 cm dorsal area of the mice was clipped and the test materials were applied over the clipped area once daily, with weekends off, for several weeks. A group of control mice that were clipped and left untreated was also included. Since a placebo cream was not provided, this study did not evaluate the effect of the placebo cream composition on hair growth.

Mice treated with the composition according to the present invention showed initial hair growth on the treated area at 2 weeks after the start of the treatment. Neither in the control nor the Minoxidil treated mice was visual hair growth seen at this time. The results were confirmed by histological analysis of Fontana-Mason stained sections of mouse skin. Hair follicles in the anogen phase of the hair cycle were observed in the mouse skin sections treated with the composition of the present invention, but not in the control or in the Minoxidil treated mice.

Further Examples Utilizing Alternative Redox Agents (Reducing Agents)

In certain aspects of the present invention, the catalyst system using transition metals (Cu or Fe) as trace catalytic metals, may require the presence of an optional redox agent (antioxidant) so as to produce H₂O₂. In order to form hydrogen peroxide, for example, it is necessary for the redox agent to reduce Cu⁺⁺ to Cu⁺. Earlier work showed that the enediols were excellent compounds to be used in the catalytic system. In expanding the number of antioxidants, the following table was assembled after experimentation, based upon reactivity with Cu, the ability to reduce CoQ₁₀ to H₂CoQ₁₀ and the appearance of the final solution (formulary consideration).

TABLE 3

| | Reduction | | |
|---|---|---|---|
| Compound | Cu⁺⁺ | Cosmetics | Reduction CoQ₁₀ |
| Lipoic Acid | yes | clear solution | Yes (180° F.) |
| Vitamin E | ? | ugly brown ppt. | — |
| Gallic acid | yes | ugly brown ppt. | — |
| Propyl gallate | yes | ugly brown ppt. | — |
| Thymol | no | — | — |
| Cysteine | yes | clear solution | yes (140° F.) |
| Quercetin | no | — | — |
| N-acetyl cysteine | yes | clear solution | yes (140° F.) |
| Caffeic acid | yes | dark brown ppt. | — |
| Glutathione | yes | clear solution | yes |
| Uric acid | yes | slightly colored ppt | yes |

The phenolic compounds (Table 3, above) either did not reduce Cu²⁺ (e.g. thymol and quercetin) or they produced a deeply colored precipitate that was not seen to be useful. Thus, the preferred reducing agents are the following:
N-acetyl cysteine
Lipoic acid (reduced)
Glutathione (reduced)
Cysteine
Uric acid

The above antioxidant compounds readily reduce CoQ₁₀ to ubiquinol. Skin tests of the above antioxidants using copper lactate as the trace metal catalyst showed swelling of the papilla (skin test previously described).

### Composition Example Formula T1 (Technology I)

Using the above antioxidants, the following superior skin care cream was prepared:

| Component | Amount | |
|---|---|---|
| Eucerin ® - Skin Renewal | 10.0 | g |
| CoQ (reduced form) | 0.1 | g |
| Glutathione (reduced) | 0.3 | g |
| Copper lactate (0.5% solution) | 0.1 | cc |

In the above cosmetic formula T1 (based upon Technology I), the CoQ₁₀ is obtained from softgel capsules made by Life Extension™. The capsules contain d-limonene which makes the soluble encapsulated components (30 mg CoQ₁₀ with 45 mg cold pressed oil from orange peel which is 90% limonene). It is self-emulsifying in water. The CoQ₁₀ is reduced to H₂CoQ₁₀ (ubiquinol) using the above antioxidants (Table 3) along with the copper lactate or iron lactate trace metal catalysts. An additional benefit is that excess antioxidant keeps it from oxidizing in air which would cause the readily bioavailable H₂CoQ₁₀ to be destroyed and produce disadvantageous colored products. The Eucerin was an important ingredient here.

The above lotion applied to the skin produces marked improvement of the skin because of the unique catalysis of dermal cells as follows:

1. The papilla is activated;

2. Sweat cells go into action (as previously described) and cause skin moisturization;

3. The oil glands are stimulated, hence, eliminating dry skin; and

4. Cell growth is stimulated causing the skin to be fuller and wrinkles are reduced.

The family of formulations made possible by the present invention and the further antioxidants, CoQ10 reduction and the inclusion of diluents and solvents, such as limonene as a mild skin irritant, produces dramatic effects on the skin. This provides a further synergistic effect on the skin.

### ADDITIONAL EXAMPLES AND USES

Cosmetic Formulation T2 (Based upon Technology II) Using Proline as Organocatalyst

| | |
|---|---|
| Proline | 2.5 g |
| Water | 1.75 g |
| Propylene Glycol | 1.75 g |
| Atrac-Tain Lotion ® | 5.00 g |
| | 10.00 g = 25% praline |

This cosmetic formulation demonstrated excellent activation of the papilla-cornium layer. Cosmetic Formulation Using Formulations T1 and T2

| | |
|---|---|
| T1 formulation (as above) | 5 g. |
| T2 formulation (as above) | 5 g. |
| | 10 g |

The above formulation produced excellent skin care results—even superior to formulation T1 alone.

### EXAMPLE

### Treatment of Acne

Since formulations T1 (based upon Technology I) and T2 (based upon Technology II) enhance dermal growth and repair biomechanisms, it is believed that each technology or both together are excellent topical treatments for acne.

### EXAMPLE

### Treatment of Keratosis

An active formulation for use in treating keratosis appears below:

| | | |
|---|---|---|
| Eucerin Skin Renewal | 9.5 | g. |
| Sodium Ascorbate (15% soluition) | 0.5 | cc |
| Fe lactate (1% solution) | 0.5 | cc |

The above formulation when applied to a large keratosis (e.g. 1.0 cm) twice daily and covered by a band-aid for up to a week in time, causes the keratosis to disintegrate. The results are unexpected.

### EXAMPLE

### Multiple Antioxidant Formulation

| | | |
|---|---|---|
| Eucerin Skin Renewal | 10.00 | g |
| Sodium ascorbate (15% sol) | 0.05 | cc |
| Copper lactate (1% sol) | 0.1 | cc |
| CoQ₁₀* reduced (ubiquionol) | 0.1 | g |
| Glutathione (reduced) | 0.3 | g |

*The source of CoQ₁₀ is from Life Extension ™ soft gels containing d-limonene as described previously. The CoQ₁₀ is reduced when it comes into contact with glutathione in the presence of the trace metal catalyst copper lactate.

1. A composition comprising, in effective amounts:
(a) at least one bio-activating organocatalyst;
(b) a pharmaceutically acceptable carrier; and the following (c)-(f) each as optional components in effective amounts:
(c) a redox agent that produces peroxide;
(d) a dermatologically acceptable transition metal-containing component;
(e) a dermatologically active enzymatic component; and
(f) a desquamation/exfoliating agent,
wherein the composition has a pH of approximately 4 to 9 and is adapted for administration to the skin or hair of an animal.

2. The composition according to claim 1 wherein said organocatalyst is selected from the group consisting of polyproline (from 100 mer and above, preferably about 100 mer to about 1000 mer), oligoproline (from 2 to about 100 mer, preferably about 2 to 10 mer) proline and its derivatives such as d,l-, d- or l-proline, d- or l-4-hydroxylproline, d or l-proline-t-butyl ester, N-acetyl-1-proline, N-acetyl-d-proline, d- or l-histidine, d- or l-phenylalanine, polyphenylalanine (from 100 mer and above, preferably about 100 mer to about 1000 mer), oligophenylalanine (from 2 to about 100 mer, preferably about 2 to 10 mer), polymeric and oligomeric mixtures (preferably polypeptides or oligopeptides) of proline and phenylalanine (from 2 to more than 1000 mer, preferably 2 to 100 mer, more preferably 2 to 10 mer), imidazolidone and its derivatives, such as 2-imidazolidone-4-carboxylic acid, quinine and its derivatives and salts such as quinine sulfate, quinine hydrosulfate, quinine HCL, quinidine and its salts including quinidine hydrochloride, quinidine sulfate, quinidine gluconate, chloral hydrate, mixtures of proline and aminobutyric acid (1-proline/aminobutyric acid as oligo or polymeric aminoacid multicomponent), mixtures of 1-proline and glycine (1-proline/glycine as oligo or polymeric or aminoacid multicomponent), pyridine derivatives including 4-dimethylaminopyridine, triazole, as well as pharmaceutically acceptable salts of any one or more of the above-described organocatalysts, thereof, where applicable, including enantiomerically pure or enriched materials, as well as racemic mixtures of these compounds.

3. The composition according to claim 1 wherein said organocatalyst is d,l-proline, d-proline, l-proline, l-4-hydroxyproline, l-proline-t-butylester, a poly- or oligopeptide comprising 1-proline and aminobutyric acid, 2-imidazolidone-4-carboxylic acid or mixtures, thereof.

4. The composition according to claim 1 wherein said organocatalyst is proline or 2-imidazolidone-4-carboxylic acid.

5. The composition of claim 1, wherein:
(a) the dermatologically active enzymatic component is an antioxidant transducer of mitochondrial oxidative phosphorylation;
(b) said redox agent is ascorbic acid, an ascorbate derivative or salt, dihydroxymaleic acid or a salt, lipoic acid or a salt, dihydrolipoic acid or a salt, cholesterol or a salt or derivative, vitamin E or its salts or derivatives, flavanones, flavone, flavanol, gallic acid, an ester or salt of gallic acid, thymol, quercetin, rutin, hydroxytyrosol, caffeic acid, ellagic acid, cysteine, N-acetyl cysteine, caffeic acid, reduced glutathione, uric acid, 2- or 3-butylated hydroxyanisole (BHA), butylated hydroxytoluene (BHT), tertiary butylated hydroquinone, homeocysteine, trolox, N,N′-Diphenyl-p-phenylene diamine, promethazine, CoQ₁₀, reduced CoQ₁₀, allopurinol, probucol, and mixtures thereof; and
(c) the dermatologically acceptable transition metal-containing component contains copper, iron, cobalt, or manganese; and
(d) the optional desquamation/exfoliating agent is a dermatologically acceptable acid or ester composition or polypeptide composition.

6. The composition of claim 2, wherein:
(a) the dermatologically active enzymatic component is CoQ₁₀ or H₂CoQ₁₀;
(b) the redox agent is ascorbic acid, an ascorbate derivative or salt or dihydroxymaleic acid or a salt, lipoic acid or a salt, dihydrolipoic acid or a salt, or cholesterol or a salt or derivative, vitamin E or its salts or derivatives, flavanones, flavone, flavanol, gallic acid, an ester or salt of gallic acid, thymol, quercetin, rutin, hydroxytyrosol, caffeic acid, ellagic acid, cysteine, N-acetyl cysteine, caffeic acid, reduced glutathione, uric acid, 2- or 3-butylated hydroxyanisole (BHA), butylated hydroxytoluene (BHT), tertiary butylated hydroquinone, homeocysteine, trolox, N,N′-Diphenyl-p-phenylene diamine, promethazine, CoQ₁₀, reduced CoQ₁₀, allopurinol, probucol, and mixtures thereof; and
(c) the dermatologically acceptable transition metal-containing component is copper histidine, ferrous histidine, ferrous EDTA, ferrous desferrioxamine, copper EDTA or mixtures thereof; and
(d) the desquamation agent is an alpha or beta hydroxy acid or mixtures thereof.

7. The composition of claim 3 wherein said acid is lactic acid, salicylic acid or mixtures, thereof.

8. The composition of according to claim 2, wherein the weight percentage ratio of redox agent to dermatologically acceptable transition metal-containing component is from approximately 250:1 to approximately 5:1.

9. The composition of claim 2, wherein the weight percentage ratio of redox agent to dermatologically acceptable transition metal-containing component is approximately 100:1 to about 6:1.

10. (canceled)

11. (canceled)

12. (canceled)

13. A composition of claim 1 comprising by weight about 1% to about 10% CoQ10, about 1%-10% lipoic acid (micronized), about 1% to 10% ascorbyl palmitate, about 80% to 95% exfoliant cream base, and about 0.2% to 1.5% copper lactate.

14. A composition of claim 1 comprising by weight about 1% to about 10% CoQ10, about 1%-10% lipoic acid (micronized), about 1% to 10% dihydroxymaleic acid, about 80% to 95% exfoliant cream base, and about 0.2% to 1.5% ferrous histidine.

15. (canceled)

16. (canceled)

17. A composition of claim 6 wherein:
1. the dermatologically active enzyme component is H₂CoQ₁₀;
2. the redox agent comprises an ascorbate derivate or salt; and
3. the dermatologically acceptable transition metal-containing component is copper histidine, ferrous histidine, ferrous EDTA, ferrous desferrioxamine or copper lactate.

18. A composition of claim 6 wherein the CoQ₁₀ is submicronized.

19. (canceled)

20. The composition according to claim 1 wherein said composition is in topical dosage form.

21. The composition according to claim 1 wherein said composition is a skin cream, lotion, emulsion or gel.

22. A composition according to claim 1 including an effective amount of a chemical irritant in place of or in addition to said redox agent and said transition-metal containing component.

23. The composition according to claim 22 wherein said chemical irritant is selected from the group consisting of ethanol, isopropanol, ammonia spirit, aromatics, creosote, eucalyptol, eucalyptus oil, green soap, irritant surfactants, tincture of pine needle oil, poplar bud, resorcinol, resorcinol ointment, resorcinol monoacetate, storax, anthralin, anthralin ointment, thymol, thyme, carvacrol, pine tar, coal tar, tar oil, ichthammol, Peruvian balsam, Arnica (wolf's bane), cantharides, chrysarobin, formic acid, Grindelia, Juniper Tar, Myrrh, capsaicin, piperine, mustard, nicotinic acid, camphor, menthol and mixtures thereof.

24. A method of treating an inflammatory disorder of the skin comprising administering to a mammal in need thereof a therapeutically effective amount of a composition according to claim 1.

25. A method of stimulating mammalian skin follicles comprising topically administering to a mammal a therapeutically effective amount of a composition according to claim 1.

26. A method of stimulating mammalian hair growth comprising topically administering to a mammal in an area of the skin or scalp where hair growth is to be stimulated a therapeutically effective amount of a composition according to claim 1.

27. The method of claim 25 wherein the mammal is a human and the composition is administered to the scalp.

28. The method of claim 26 wherein the mammal is a human and the composition is administered to the scalp.

29. A method of treating a wound in the skin of a mammal comprising applying to said wound an effective amount of a composition according to claim 1.

30. The method according to claim 29 wherein said wound is a burn, cut, scrape, scratch, minor irritation or surgical wound.

31. A method of treating damaged skin comprising applying to said skin an effective amount of a composition according to claim 1.

32. A method of treating acne comprising applying to skin affected by acne an effective amount of a composition according to claim 1.

33. A method of treating wrinkles comprising applying to wrinkled skin an effective amount of a composition according to claim 1.

34. A method of treating skin to enhance its smoothness comprising applying to said skin a composition according to claim 1.

35. A method of treating acne in affected keratinous tissue of a patient in need thereof comprising topically administering to said affected keratinous tissue of said patient a composition comprising, in effective amounts:
(a) at least one redox agent that produces peroxide;
(b) a dermatologically acceptable transition metal-containing component;
(c) a carrier;
(d) optionally, a dermatologically active enzymatic component; and
(e) optionally, a desquamation/exfoliating agent,
wherein the composition has a pH of approximately 4 to 9.

36. (canceled)

37. (canceled)

38. A method of treating sore throat, stomach ulcers, cancer, cardiovascular diseases and disease states, kidney failure and end-stage renal disease in a patient in need of therapy said method comprising administering an effective amount of a composition according to claim 1 to said patient.

39. A method of treating, rebuilding and/or repairing damage to tissues in a patient caused by infectious disease comprising administering to said patient an effective amount of a composition according to claim 1 to said patient.

40. A method of reducing CoQ₁₀ to H₂CoQ₁₀ comprising the steps of exposing CoQ₀ to a reducing agent in the presence of a dermatologically acceptable transition metal-containing component.
